(19) United States
(12) Patent Application Publication
(10) Pub. No.: US 2022/0371011 A1
(43) Pub. Date: Nov. 24, 2022

(54) Sealed Container Comprising a Device Allowing Removable Connection to an Enclosure

(71) Applicant: ABC TRANSFER, Tours (FR)

(72) Inventors: Julien FELIX, Tours (FR); Jean-Luc SCHNEIDER, Sligo (FR); Thierry GIRARD, Tours (FR)

(21) Appl. No.: 17/787,513

(22) PCT Filed: Dec. 18, 2020
(86) PCT No.: PCT/FR2020/052548

§ 371 Date: Jun. 20, 2022

(30) Foreign Application Priority Data
Dec. 20, 2019 (FR) FR1915376

(51) Int. Cl. B01L 3/00; B65D 45/32

(52) U.S. Cl. CPC B01L 3/50825; B65D 45/325; B01L 2200/141; B01L 2300/04; B01L 2300/12; B01L 2200/026

(57) ABSTRACT

The invention relates to a sealed container (1) comprising a container body (2) having a through-hole (8) that is delimited by a flange and is closed by a removable door (7) mounted on the flange, an annular seal (13) located between the flange and the door (7), the flange, the door (7) and the seal forming a sealed connection device that can be connected to a complementary connection device on a sterile enclosure by means of bayonet connection means in order to allow sterile communication between the sealed container and the sterile enclosure, characterized in that the seal (13) comprises, on its inner peripheral circumference, an annular attachment bead (130) for attaching the door (7) to the flange inserted into a notch (70) on the door (7).

### TECHNICAL FIELD OF THE INVENTION

The invention relates to the field of containers intended to be connected in a sealed manner to sterile enclosures so as to allow their respective volumes to be placed in communication without contact with said external environment.

The invention relates more particularly to a sealed container comprising a container body having a through-hole delimited by a flange and closed off by a removable door mounted on the flange, an annular seal located between the flange and the door, the flange, the door and the seal forming a sealed connection device capable of being connected to a complementary connection device of a sterile enclosure by means of bayonet connection means in order to allow sterile communication between the sealed container and the sterile enclosure.

The sealed container according to the invention is intended in particular, but not exclusively, for the transfer of dangerous products such as certain pharmaceutical, biotechnological, biological, chemical or radioactive products, the transfer of components such as stoppers, bottles, pistons, syringes, etc., the transfer of environmental control devices such as culture medium racks, particle counters, etc., the transfer of cleaning systems, the transfer of liquids, powders, tools, the transfer of waste to the outside of the enclosure and/or the transfer of any element necessary for the production or maintenance of the production line.

### PRIOR ART

In a known manner, the connection of a sealed container to a sterile enclosure for the transfer of products, without breaking the connection and the seal, is carried out using two connection devices, one equipping the container and the other equipping the enclosure. Each connection device comprises a flange respectively delimiting the through-hole in the internal volume of the container or of the enclosure, each of the through-holes being closed off by a door. The flange and the door of the container are capable of being connected respectively to the flange and to the door of the enclosure by a bayonet connection under the action of a rotational movement of the associated flange and door of the container with respect to the flange and door of the enclosure to which they are attached. Thus, the flanges and the doors, respectively, of the container and of the enclosure are respectively provided with notches and with internal or external lugs. During the rotational movement, the container door is uncoupled from the associated flange.

These connection devices are on the whole satisfactory. However, the friction generated during the rotational movement carried out to lock the door to the flange has the drawback of producing particles that are capable of inducing contamination within the enclosed volumes, but also of producing a break in the seal of the connection. Furthermore, the internal lugs of the flange ensuring the grip of the door extend, once the door is disengaged from these lugs, into the through-hole, so that the transfer of the components may sometimes be hampered by these lugs.

The invention aims to remedy these problems by proposing a sealed container limiting the emission of particles liable to generate contamination of the enclosed volumes during the assembly of the door to the associated flange of the container.

Another object of the invention is to provide a sealed container offering an optimized through-hole as well as stable and durable sealing between the container and the enclosure to which it is connected.

### SUBJECT MATTER OF THE INVENTION

To this end, the invention proposes a sealed container comprising a container body having a through-hole delimited by a flange and closed off by a removable door mounted on the flange, an annular seal located between the flange and the door, the flange, the door and the seal forming a sealed connection device capable of being connected to a complementary connection device of a sterile enclosure by means of bayonet connection means in order to allow sterile communication between the sealed container and the sterile enclosure, the container being remarkable in that the container comprises an annular attachment bead for attaching the door to the flange inserted into a notch on the door provided for this purpose.

The door is thus assembled to the flange of the container by simple clipping, and in the same way is disassembled by unclipping.

By thus avoiding the rotational movement imposed by the bayonet connection implemented in the containers of the prior art, the production of contamination-generating particles is avoided. The attachment bead also has the advantage of taking up less space than the lugs of the flanges. This thus makes it possible to offer an increased passage diameter and thus to facilitate the passage of the components and to reduce the risks of damage to the latter during their transfer.

According to a particular embodiment, the annular attachment bead is provided on the inner peripheral circumference of the flange.

According to another embodiment, the annular attachment bead is offset on the seal. Thus, said bead is provided on the inner peripheral circumference of said seal. The attachment of the door on the flange is thus carried out by means of the seal.

Advantageously, the flange consists of two separate coaxial flange bodies, one of the flange bodies defining an external flange body arranged to allow connection of the container body to the complementary enclosure, the other flange body defining an internal flange body, said flange bodies being arranged relative to each other to define therebetween an annular interstitial space in which the seal is housed.

Advantageously, the annular attachment bead is provided on the inner peripheral circumference of the internal flange body. In the case, however, where the annular attachment bead is offset on the seal, the latter and the internal flange body are advantageously arranged so that the attachment bead rests on the front end of the internal flange body.

Advantageously, the container comprises anti-rotation means preventing the rotation of the door (7) on the flange. According to a preferred variant, the bead has a profile arranged to ensure the prevention of the rotation of the door on the flange.

Advantageously, the seal has a notched face portion intended to be in contact with the door.

Advantageously, the seal comprises means of reciprocal association with the flange.

Advantageously, the seal has at least one contact face with at least one of the flange bodies provided with a notching cooperating with a complementary notching provided on the face of said flange body opposite which it is placed, the notching of the contact face of the seal and the complementary notching forming the means of reciprocal association.

Advantageously, the external flange body comprises an internal peripheral rim abutting which the seal is positioned.

Advantageously, the container body is formed by a flexible film fixed to the flange, the flexible film being held in engagement between the external flange body and the internal flange body.

It may also be provided that the external flange body has a lower skirt comprising longitudinal slots extending from the lower end edge of the external flange body and evenly distributed over the periphery of said flange body so as to form elastic tabs capable of exerting a clamping force on the internal flange body. Provision may also be made for the container to comprise a clamping ring for clamping the external flange body on the internal flange body arranged to grip the parts of the external flange body and of the internal flange body between which the flexible film is interposed. Advantageously, the container comprises an additional annular seal housed in a peripheral groove provided on the inner face of the external flange body, the flexible film extending between the additional annular seal and the internal flange body. These features, taken alone or in combination with each other, make it possible to improve the mechanical strength of the container and to ensure optimum sealing. They also allow a container to be produced simply and quickly, without risk of deterioration of the film and contamination of the latter's contents.

Advantageously, all or part of the container is made of a bacteriostatic material.

### BRIEF DESCRIPTION OF THE DRAWINGS

Other features and advantages of the invention will become apparent from the following detailed description of the invention with reference to the appended figures, in which:

FIG. 1 shows a schematic front view of a container according to the invention;

FIG. 2 shows a longitudinal cross-sectional view of the container of FIG. 1;

FIG. 3 shows a detail view of the container of FIG. 2;

FIG. 4 shows a partial view of the external flange body of the connection device of the container of FIG. 2;

FIG. 5 shows the container of FIG. 2 provided with a drop system for component transfer;

FIG. 6 shows a detail view of the container of FIG. 5;

FIG. 7 shows a longitudinal cross-sectional view of another embodiment of a container according to the invention.

For greater clarity, identical or similar elements of the various embodiments are denoted by identical reference signs in all of the figures.

### DETAILED DESCRIPTION OF THE INVENTION

In relation to FIGS. 1 to 6, a container 1 is described according to a first embodiment of the invention. The illustrated container 1 comprises a flexible film 2 arranged to define an internal volume 3 and a through-hole allowing the internal volume 3 to be placed in communication with the exterior of the film.

The container also comprises a sealed connection device that is arranged to allow the flexible film 2 to be connected to a complementary connection device of a sterile enclosure. The connection is made in such a way as to ensure sterile communication between the internal volume 3 of said container 1 and that of the enclosure. The connection device comprises a flange on which the flexible film is attached, said flange delimiting the through-hole of the flexible film. It further comprises a removable door 7 attached to the flange so as to close the through-hole.

According to the invention, the flange is constituted by two mutually different pieces, a first piece forming a so-called external flange body and a second piece forming a so-called internal flange body, between which the flexible film extends.

The internal flange body 6 has a notched zone adjacent to the bearing recess arranged to cooperate with a complementary notching provided on the inner face of the external flange body 5.

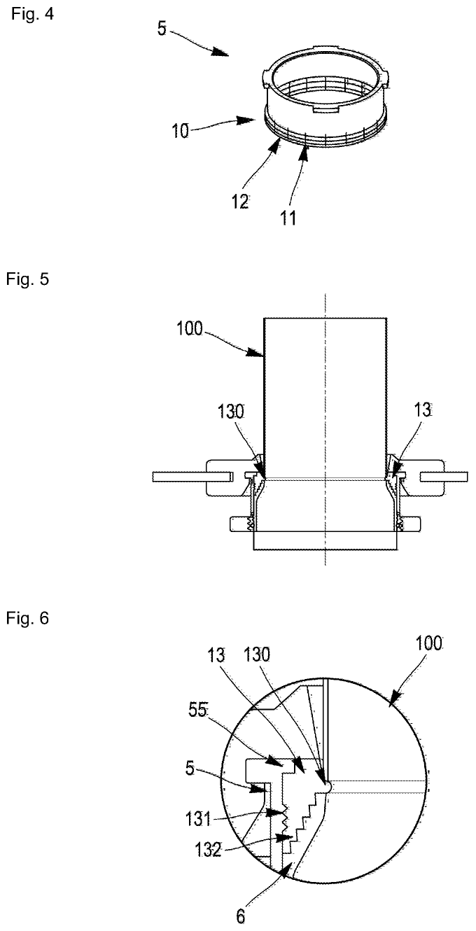

According to an advantageous configuration illustrated in FIG. 4, the external flange body 5 comprises a lower skirt 10 comprising longitudinal slots 11 extending from the lower end edge of said flange body. These longitudinal slots 11 are advantageously evenly distributed over the periphery of said flange body. They thus delimit elastic tabs 12 capable of exerting a uniform clamping force over the entire outer periphery of the internal flange body 6 when the external flange body 5 is mounted on the internal flange body 6. The elastic tabs 12 comprise means of reciprocal association with clamping means, described below, and the internal flange body 6, respectively. In the illustrated example, the means of reciprocal association with the clamping ring consist of a thread and a notching. The clamping means are arranged to clamp the parts of the external flange body 5 and of the internal flange body 6 between which the flexible film 2 is interposed. In the illustrated example, the clamping means are in the form of an external clamping ring comprising an internal thread with a shape complementary to a thread provided on the outer face of the external flange body 5.

The container 1 further comprises a first annular seal 8 housed in a peripheral groove formed on the inner face of the external flange body 5 and the outer face of the internal flange body 6. As shown in FIG. 3, the flexible film 2 is kept gripped between the annular seal 8 and the internal flange body 6. In order to improve the retention of the flexible film 2 between the two flange bodies constituting the flange, the internal flange body 6 comprises an outer face provided with a bearing recess located opposite the peripheral groove of the external flange body 5. Thus, when the external flange body 5 is clamped on the internal flange body 6, the annular seal 8 crushes the flexible film 2 at the bearing recess, ensuring improved engagement of the flexible film 2.

The container 1 comprises a second annular seal 13 that is borne by the flange. It defines a door/flange seal. When the door is in the position closing off the through-hole, the annular seal 13 is housed in the space delimited by the external flange body 5, the internal flange body 6 and the door 7. Advantageously, the annular seal 13 has a cross-section of shape matching the area in which it is housed. In the illustrated embodiment, the annular seal 13 has a substantially triangular section.

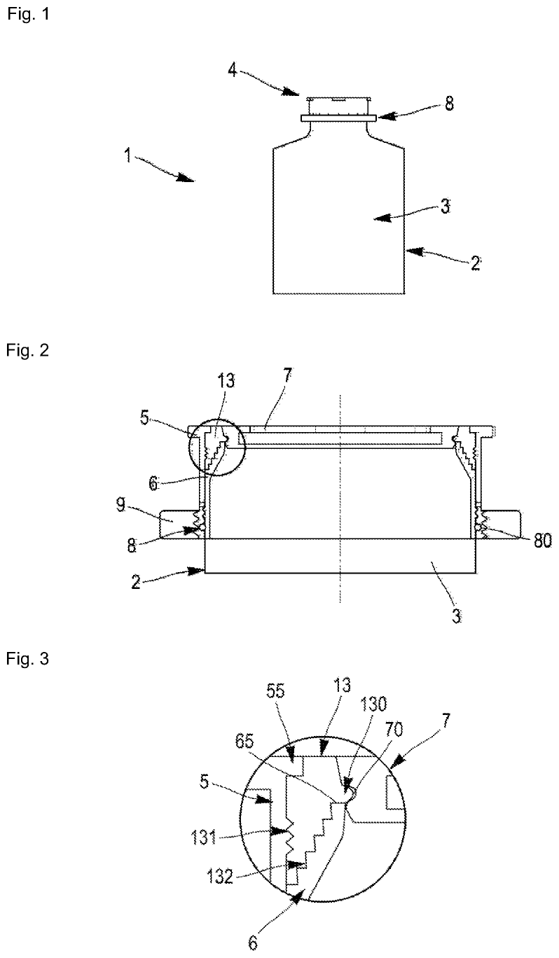

As illustrated in FIGS. 2 and 3, and according to the invention, the door 7 is held on the flange by means of the seal 13. To this end, it has, on its inner peripheral circumference, an annular attachment bead 130 that is capable of being inserted into a notch 70 made on the outer peripheral edge of the door 7.

In order to prevent the attachment bead 130 from collapsing, the seal 13 is arranged with the internal flange body 6 so that the attachment bead 130 rests on the front end 65 of the internal flange body 6.

In order to ensure that the seal 130 is held between the external and internal flange bodies 5, 6, the latter comprises means of reciprocal association with the external flange body 5 on the one hand and the internal flange body 6 on the other hand. In the illustrated embodiment, the means of reciprocal association are in the form of notched zones. Thus, the faces of the seal in contact with the external flange body 5 and the external flange body 6 comprise a notching 131, 132 cooperating with a complementary notching formed on the faces of said flange bodies opposite which they are placed. In the example illustrated, the contact face 132 with the internal flange body in particular has a stepped shape in order to ensure reinforced holding of the seal 13 between the external and internal flange bodies 5, 6. Advantageously, the external flange body 5 is provided with an internal peripheral rim 55 abutting which the seal 13 is positioned, thus ensuring axial holding of said seal, in particular when the door 7 is removed. The seal 13 and its assembly with the flange according to the arrangement described above thus allow elimination of any risk of deformation, torsion and displacement of the seal 13 during the production of the connection device as well as during the use of the container.

Advantageously, the attachment bead 130 is arranged to allow docking of a drop system 100 for the component transfer. As illustrated in FIGS. 5 and 6, the latter is mounted to bear against the attachment bead 130.

Due to the arrangement of its component elements, the connection device is assembled by simple clipping of the elements. To make the container 1, the part of the flexible film located on the side of the through-hole will be arranged so as to be placed between the external flange body 5 and the internal flange body 6. The clamping will be effective by clamping the ring 9 on the external flange body 6, causing the tabs 12 to be gripped against the internal flange body 6. The flexible film 2 may be disassembled by unscrewing the clamping ring 9 and unclipping the external flange body 6 from the internal flange body 7.

The flexible film 2 and the connection device are advantageously made of a bacteriostatic material. The seals 8 and 13 are made of a silicone material or other materials minimizing remanence phenomena. Provision may also be made for the seals to be made of a bacteriostatic material.

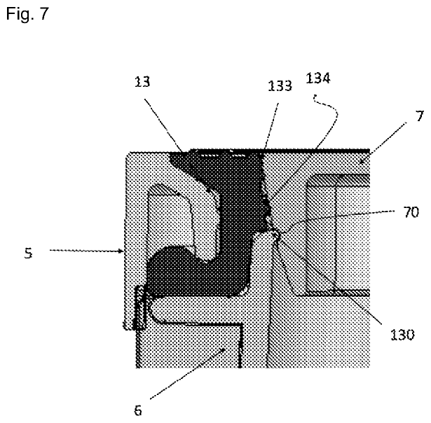

FIG. 7 illustrates another embodiment of mounting the door 7 on the flange. In this embodiment, the flange also comprises an external flange body 5 and an internal flange body 6. The annular attachment bead 130 is, however, borne by the internal flange body. Advantageously, the face portion 133 of the seal 13 intended to be in contact with the door 7 has a notching 134. The term “notching” means any shape making the face of the seal non-flat so as to promote friction. The term “notching” thus by extension comprises scoring, aliasing, bumping, etc. Such an arrangement of the seal with the annular attachment bead 130 borne by the flange 7 has the advantage of improving the holding of the door 7 on the flange and jointly of reinforcing the seal between the door 7 and the internal flange body 6.

The invention is described in the foregoing by way of example. It is understood that a person skilled in the art is in a position to produce various variant embodiments of the invention without thereby departing from the scope of the invention.

1. A sealed container comprising:
a container body having a through-hole delimited by a flange and closed by a removable door mounted on the flange,
an annular seal located between the flange and the door,
the flange, the door and the seal forming a sealed connection device capable of being connected to a complementary connection device of a sterile enclosure by means of bayonet connection means in order to allow sterile communication between the sealed container and the sterile enclosure,
characterized in that the container comprises an annular attachment bead for attaching the door to the flange inserted into a notch of said door provided for this purpose, so as to allow an assembly of the door on the flange by clipping, and vice versa.

2. The sealed container according to claim 1, characterized in that the annular attachment bead is provided on the inner peripheral circumference of the flange.

3. The sealed container according to claim 1, characterized in that the annular attachment bead is provided on the inner peripheral circumference of the seal.

4. The sealed container according to claim 1, characterized in that the flange consists of two separate coaxial flange bodies, one of the flange bodies defining an external flange body arranged to allow connection of the container body to the complementary enclosure, the other flange body defining an internal flange body, said flange bodies being arranged relative to each other to define therebetween an annular interstitial space in which the seal is housed.

5. The sealed container according to claim 4, characterized in that the annular attachment bead is provided on the inner peripheral circumference of the internal flange body.

6. The sealed container according to claim 4, characterized in that the annular attachment bead is provided on the inner peripheral circumference of the seal and the seal and the internal flange body are arranged so that the attachment bead rests on the front end of the internal flange body.

7. The sealed container according to claim 1, characterized in that it comprises anti-rotation means preventing the rotation of the door on the flange.

8. The sealed container according to claim 1, characterized in that the seal has a notched face portion intended to be in contact with the door.

9. The sealed container according to claim 1, characterized in that the seal comprises means of reciprocal association with the flange.

10. The sealed container according to claim 4, characterized in that the seal has at least one contact face with at least one of the flange bodies, said contact face being provided with a notching cooperating with a complementary notching provided on the face of said flange body opposite which it is placed, the notching of the contact face of the seal and the complementary notching forming the means of reciprocal association.

11. The sealed container according to claim 4, characterized in that the external flange body comprises an internal peripheral rim abutting which the seal is positioned.

12. The sealed container according to claim 4, characterized in that the external flange body comprises a lower skirt comprising longitudinal slots extending from the lower end edge of the external flange body and evenly distributed over the periphery of said flange body so as to form elastic tabs capable of exerting a clamping force on the internal flange body.

13. The sealed container according to claim 4, characterized in that the container body is formed by a flexible film fixed to the flange, the flexible film being held in engagement between the external flange body and the internal flange body.

14. The sealed container according to claim 13, characterized in that it comprises a clamping ring for clamping the external flange body on the internal flange body arranged to grip the parts of the external flange body and of the internal flange body between which the flexible film is interposed.

15. The sealed container according to claim 13, characterized in that it comprises an additional annular seal housed in a peripheral groove provided on the inner face of the external flange body, the flexible film extending between the additional annular seal and the internal flange body.

16. The sealed container according to claim 1, characterized in that all or part of the container is made of a bacteriostatic material.
